(19) United States
(12) Patent Application Publication
(10) Pub. No.: US 2024/0190799 A1
(43) Pub. Date: Jun. 13, 2024

(54) CHEMICAL UPCYCLING OF HYDROXYLATED POLYMERS VIA C-C BOND CLEAVAGE REACTIONS

(71) Applicants: THE TRUSTEES OF PRINCETON UNIVERSITY, Princeton, NJ (US); CORNELL UNIVERSITY, Ithaca, NY (US)

(72) Inventors: Robert R. Knowles, Princeton, NJ (US); Suong T. Nguyen, Princeton, NJ (US); Elizabeth A. Mcloughlin, Plainsboro, NJ (US); James H. Cox, Princeton, NJ (US); Brett P. Fors, Ithaca, NY (US)

(21) Appl. No.: 18/285,087

(22) PCT Filed: Mar. 31, 2022
(86) PCT No.: PCT/US2022/022768

§ 371 Date: Sep. 29, 2023

Related U.S. Application Data
(60) Provisional application No. 63/168,849, filed on Mar. 31, 2021.

(51) Int. Cl. C07C 41/18; C07C 1/22; C07C 29/00; C07C 37/055; C07C 45/55; C07C 51/00; C07C 253/00; C08G 63/16; C08G 69/00

(52) U.S. Cl. CPC C07C 41/18; C07C 1/22; C07C 29/00; C07C 37/055; C07C 45/55; C07C 51/00; C07C 253/00; C08G 63/16; C08G 69/00

(57) ABSTRACT

In one aspect, methods of depolymerization are described herein comprising providing a synthetic polymer including a hydroxylated aliphatic backbone or hydroxylated backbone segments, and homolytically activing O—H bonds of the hydroxyl groups. Homolytic activation induces the formation of alkoxy radical intermediates followed by C—C bond β-scission events breaking the polymer backbone into depolymerization products. In some embodiments, depolymerization products comprise alkyl radical intermediates reduced by hydrogen atom transfer. Moreover, in some embodiments, the depolymerization products are further reacted into difunctionalized products or comprise functionalities derived from the polymer structure. The difunctionalized products can subsequently be employed in polymerization processes for the production of additional synthetic polymers.

### RELATED APPLICATION DATA

The present application claims priority pursuant to Article 8 of the Patent Cooperation Treaty to U.S. Provisional Patent Application Ser. No. 63/168,849 filed Mar. 31, 2021, which is hereby incorporated by reference in its entirety.

### STATEMENT OF GOVERNMENT RIGHTS

This invention was made with government support under Grant No. DE-SC0019370 awarded by the Department of Energy and Grant No. DGE-2039656 awarded by the National Science Foundation. The government has certain rights in the invention.

### FIELD

The present invention related to compositions and methods for polymer recycling and, in particular, to compositions and methods for depolymerizing hydroxylated synthetic polymers into useful feedstocks for subsequent polymer productions.

### BACKGROUND

Single-use plastics have revolutionized life across all sectors of society. While the low cost, durability, and chemical stability of these materials have led to a dramatic increase in there production and use, these same properties have prompted serious concerns about the long-term environmental challenges associated with plastic pollution and sustainability. A transition away from single-use plastics and towards a more sustainable and circular polymer economy is now widely viewed as being imperative on both environmental and economic grounds.

Contemporary recycling of plastics on an industrial scale relies primarily on mechanical reprocessing, which frequently results in products of diminished quality compared to the original and cannot be applied to all plastics. A more ideal end-of-life treatment for post-consumer plastics entails chemically transforming the polymers back into virgin monomers or other well-defined small molecules feedstocks that can then be repolymerized into recyclable plastics. While appealing in principle, chemical recycling to monomer is generally limited to the hydrolysis of condensation polymers (e.g., polyesters, polycarbonates, and polyamides) and the pyrolysis of certain polymers that lack reactive functional groups along their backbone (e.g., polystyrene). Such limitations necessarily preclude recycling of polyolefins, including polyethylene and polypropylene, which constituted a substantial portion of plastic waste.

### SUMMARY

The foregoing disadvantages are addressed by methods described herein, which can enable efficient depolymerization via C—C bond cleavage at locations in the polymer backbone. In one aspect, a method of depolymerization comprises providing a synthetic polymer including a hydroxylated aliphatic backbone or hydroxylated backbone segments, and homolytically activating O—H bonds of the hydroxyl groups. Homolytic activation induces the formation of alkoxy radical intermediates followed by C—C bond β-scission events breaking the polymer backbone into depolymerization products. In some embodiments, depolymerization products comprise alkyl radical intermediates reduced by a hydrogen atom transfer catalyst. Moreover, in some embodiments, the depolymerization products are converted into difunctionalized products containing functionality not originally present in the polymer or, alternatively, maintaining those derived from the polymer. The difunctionalized products can subsequently be employed in repolymerization processes for the production of additional synthetic polymers.

In some embodiments, hydroxyl groups pendant to the aliphatic backbone or backbone segments are intrinsic to the synthetic polymeric species undergoing depolymerization by methods described herein. Alternatively, the polymeric species may not include pendant hydroxyl groups. In such embodiments, the aliphatic backbone or backbone segments may be hydroxylated to render the synthetic polymeric species compatible with depolymerization methods described herein. In this way, polyolefins such as polyethylene and polypropylene can be hydroxylated and subsequently depolymerized.

These and other embodiments are further described in the following detailed description.

### BRIEF DESCRIPTION OF THE DRAWINGS

FIG. 1A illustrates a general mechanism for depolymerization of hydroxylated synthetic polymers according to some methods described herein.

FIG. 1B illustrates various hydroxyl containing thermoplastics amenable to depolymerization according to methods described herein.

FIG. 1C illustrates depolymerization of PVA and EVOH according to some embodiments of methods described herein.

FIG. 1D illustrates hydroxylation of a polyolefin backbone rendering the polyolefin suitable for depolymerization according to some embodiments.

FIG. 2 illustrates examples of epoxy resins and associated curing agents for the synthesis of epoxy thermosets amenable to depolymerization according to methods described herein.

FIG. 3A illustrates degradable-by-design hydroxylated polymeric species for a circular polymer economy according to some embodiments.

FIG. 3B illustrates a synthetic pathway to hydroxylated polymeric species via ring opening metathesis according to some embodiments.

FIG. 4 illustrates depolymerization and recycling of mixed plastic waste streams comprising hydroxylated polymers according to methods described herein.

FIG. 5A illustrates incorporating functionalities into monomer structure according to some embodiments.

FIG. 5B illustrates introducing functionalities into depolymerization product via trapping of the intermediate radical according to some embodiments.

FIG. 6A illustrates control of carbon chain length in depolymerization products according to some embodiments.

FIG. 6B illustrates controlling end-group identity of polymers through catalyst and termination agent design according to some embodiments.

FIG. 7A illustrates various hydroxylated ring opening metathesis polymerization monomers that would provide access to hydroxylated polymers according to some embodiments.

FIG. 7B illustrates acyclic diene metathesis monomers according to some embodiments.

FIG. 8 illustrates a strategy for photocatalytic depolymerization of hydroxylated polymers according to methods described herein.

FIG. 9 provides study parameters and results for the photocatalytic depolymerization of phenoxy resin according to methods described herein.

FIG. 10A illustrates photocatalytic recycling of commercial phenoxy thermoplastic to bisphenol A. Depolymerization reactions were performed on 0.5-mmol —OH (142 mg) scale. Yields were reported for isolated and purified materials and are the average of two experiments.

FIGS. 10B and 10C are GPC traces and GC measurements showing the successful depolymerization of phenoxy and the increase in the GC yield of M₁ over the course of 24 h.

FIG. 10D illustrates selective depolymerization of phenoxy resin in the presence of other commodity plastics. Depolymerization reactions were performed on 0.05 mmol —OH scale (14.2 mg phenoxy resin) with an equal mass of each polymer additive. Yields were determined by GC analysis of crude reaction mixtures relative to an internal standard.

FIG. 11 are ¹H NMR spectra (in DMSO-d₆) of PMP homopolymer before irradiation (spectrum was taken at 100° C.) and after irradiation (spectrum was taken at ambient temperature). NMR analysis of the post-irradiation mixture showed the absence of Hₐ corresponding to the original polymer and the presence of H_{a′} corresponding to the aldehyde products, indicating complete polymer degradation.

FIG. 12A illustrates photocatalytic depolymerization reactions of PMP and OTBS homopolymers. Reactions were performed on 0.5-mmol —OH scale, which corresponds to 117 mg and 129 mg of PMP and OTBS homopolymer, respectively. Yields of each monomeric product were determined by GC (M₂, M₃, M₅ and M₆) or HPLC (M₄) analysis of the crude reaction mixtures relative to an internal standard and are the average of two experiments.

FIG. 12B provides derivatization of depolymerization products into useful chemical feedstocks, according to some embodiments.

FIGS. 13A and 13C provide thermal and mechanical properties of PMP-x copolymers, respectively.

FIGS. 13B and 13D provide thermal and mechanical properties of OTBS-x copolymers, respectively.

FIG. 14A illustrates the structures of various epoxy resins and thiols according to some embodiments.

FIG. 14B illustrates synthesis of thiol epoxies according to some embodiments.

FIG. 15 illustrates epoxy resin depolymerization according to some embodiments of methods described herein.

### DETAILED DESCRIPTION

Embodiments described herein can be understood more readily by reference to the following detailed description and examples and their previous and following descriptions. Elements, apparatus, and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

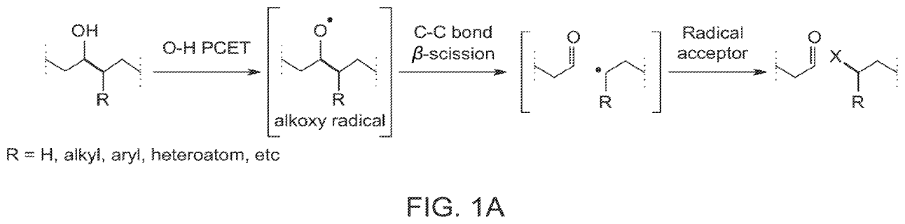

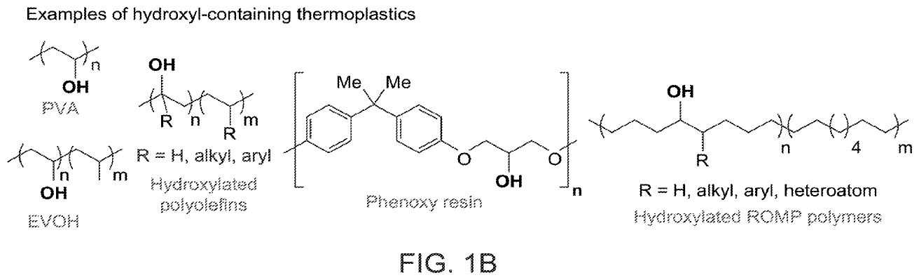

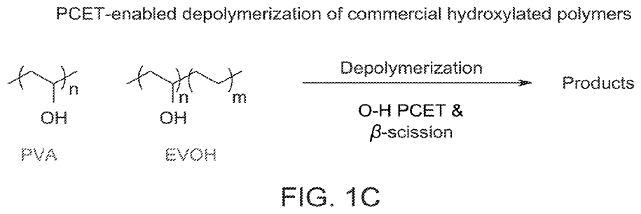

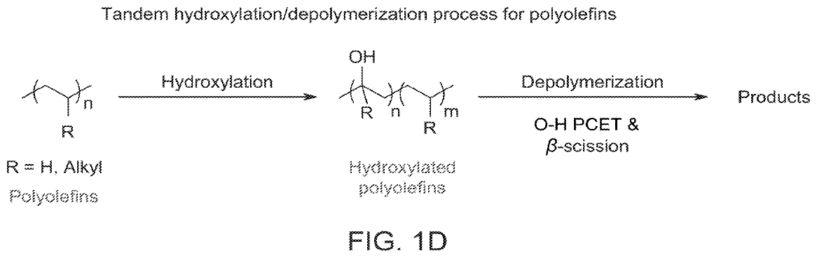

Methods of depolymerization are provided. In one aspect, a method comprises providing a synthetic polymer including a hydroxylated aliphatic backbone or hydroxylated backbone segments and homolytically activating O—H bonds of the hydroxyl groups. Homolytic activation induces the formation of alkoxy radical intermediates followed by C—C bond β-scission events breaking the polymer backbone into depolymerization products. As used herein, synthetic polymer covers synthetic homopolymers and synthetic copolymers. FIG. 1A illustrates a general mechanism for depolymerization of hydroxylated synthetic polymers according to some methods described herein.

Turning now to specific components, the synthetic polymer undergoing depolymerization comprises a hydroxylated aliphatic backbone or hydroxylated backbone segments. A hydroxylated backbone segment can correspond to a single monomeric species of the synthetic polymer or a plurality of linked monomeric species of the synthetic polymer. The synthetic polymer backbone can exhibit any degree of hydroxylation and/or spacing of hydroxyl groups along the backbone consistent with the technical objectives described herein. In some embodiments, the degree of hydroxylation and/or hydroxyl group spacing is dependent on the specific identity of the synthetic polymer or copolymer undergoing depolymerization. Suitable synthetic polymers include thermoplastics as well as thermosets. As an example, the backbone of polyvinyl alcohol (PVA) will exhibit a differing degree/spacing of hydroxylation relative to ethylene vinyl alcohol (EVA). Similarly, the degree/spacing of hydroxylation of the poly(hydroxyl ether) of bisphenol A will differ from that of PVA and EVA. Hydroxyl groups pendant to the aliphatic backbone or backbone segments are intrinsic to the polymeric species undergoing depolymerization. FIG. 1B illustrates various hydroxyl containing thermoplastics operable for depolymerization according to methods described herein. FIG. 1C illustrates depolymerization of PVA and EVOH according to some embodiments of methods described herein. In some embodiments, spacing of the hydroxyl groups can be employed to tune the carbon count of the depolymerization products.

Alternatively, the synthetic polymeric species may not include pendant hydroxyl groups. In such embodiments, the aliphatic backbone or backbone segments may be hydroxylated to render the polymeric species compatible with depolymerization methods described herein. In this way, polyolefins such as polyethylene, polypropylene, and polystyrene can be hydroxylated and subsequently depolymerized. Use of Ni(II) based catalyst and mCPBA as an oxidant, for example, can introduce hydroxyl groups along the polyethylene backbone. FIG. 1D illustrates hydroxylation of a polyolefin backbone rendering the polyolefin suitable for depolymerization according to some embodiments.

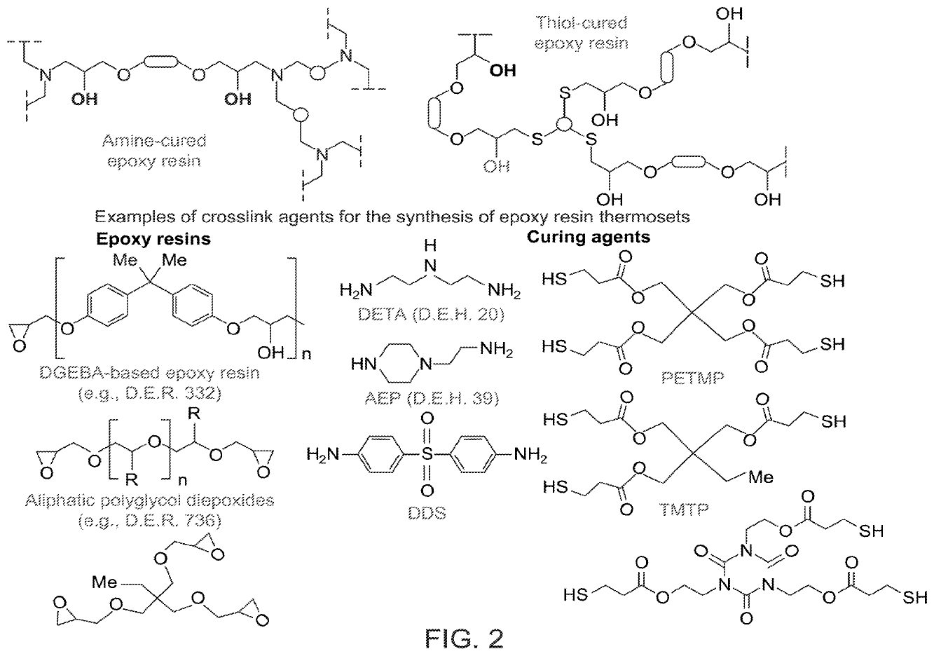

In addition to thermoplastics, the synthetic polymer undergoing depolymerization can be a thermoset comprising hydroxylated backbone or hydroxylated backbone segments. The hydroxylated backbone or hydroxylated backbone segments may exist in main chains of the thermoset and/or in crosslinking portions of the thermoset. FIG. 2 illustrates examples of epoxy resins and associated curing agents for the synthesis of epoxy thermosets amenable to depolymerization according to methods described herein.

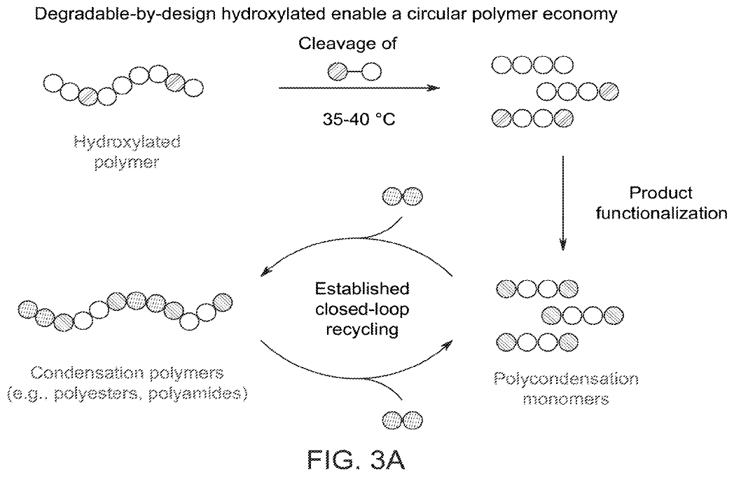

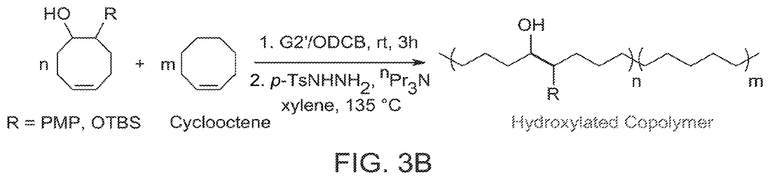

Moreover, many commercial polymers are not recycled in a closed-loop manner because of the paucity of selective and efficient depolymerization methods. One way to establish a circular polymer economy is to design new polymers that can be degraded into high-value chemical products under relatively mild conditions. For these next-generation materials to replace nondegradable plastics, they need to have properties that are similar or superior to the existing materials. Seeking to address this challenge, embodiments herein aim to design degradable polyolefin-like materials given that polyolefins are the largest class of polymers by global production. By incorporating a hydroxyl group within the cyclooalkene monomer, it is possible to create polyolefin mimics that can be degraded by the present methods. FIG. 3A illustrates degradable-by-design hydroxylated polymeric species for a circular polymer economy according to some embodiments. These materials can be synthesized through a two-step ring-opening metathesis polymerization (ROMP) and hydrogenation sequence, as illustrated in FIG. 3B. Additionally, ROMP offers opportunities to rapidly generate a library of materials with diverse thermomechanical properties via the modification of the monomer structure and/or copolymerization conditions.

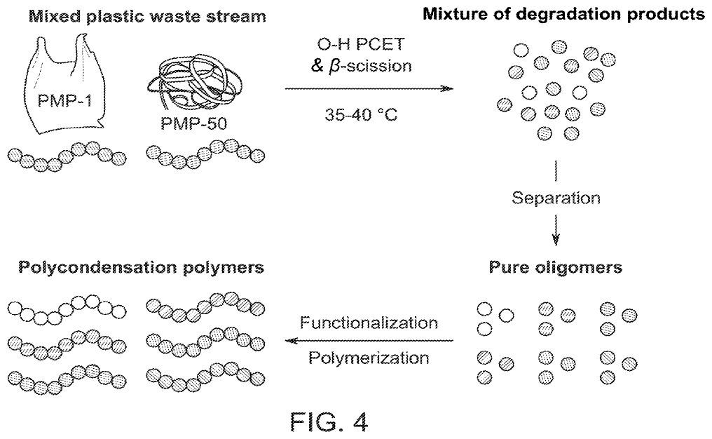

Due to the selective pathway and mechanisms detailed further herein, depolymerization of hydroxylated synthetic polymer is not inhibited by the presence of other types of polymeric materials. Polymeric materials lacking hydroxylated backbone or hydroxylated backbone segments are not depolymerized or degraded by the present methods. Therefore, hydroxylated synthetic polymeric materials can be selectively depolymerized in the presence of other polymeric species. Such selective depolymerization can assist in efficient recycling of multilayered polymeric films or other architectures where combinations or mixtures of differing polymer species are used. For example, due to its superior gas barrier properties, EVOH is frequently used in multilayer co-extruded films with high-density polyethylene (HDPE) and polypropylene (PP). Recycling these materials is often problematic as the EVOH layer tends to clump together when reprocessed and thus results in holes in the recycled PE/PP films. Methods described herein can be used to selectively degrade the EVOH layer in the presence of these polyolefins, thereby addressing a significant problem associated with recycling multilayered plastic materials. Additionally, since hydroxylated polymers and copolymers can be depolymerized by the same mechanism described herein, complex mixtures of these hydroxylated polymers and/or copolymers can be depolymerized together in a single waste stream, thereby eliminating the need to separate distinct types of plastic waste. FIG. 4 illustrates depolymerization and recycling of mixed plastic waste streams comprising hydroxylated polymers according methods described herein.

As described herein, O—H bonds of the pendant hydroxyl groups are homolytically activated, inducing the formation of alkoxy radial intermediates. Any method of homolytic activation of the O—H bonds consistent with the technical objectives presented herein can be employed. In some embodiments, the homolytic activation occurs via proton-coupled electron transfer (PCET). The proton-coupled electron transfer can be concerted or stepwise. To facilitate or enhance the depolymerization mechanism, the aliphatic backbone or backbone segments can comprise pendant radical stabilizing moieties. The radical stabilizing moieties can be located at a β-positon to the hydroxyl groups. In some embodiments, the pendant radical stabilizing moieties are selected from the group consisting of aryl, heteroaryl, and heteroatom. Radical stabilizing species, for example, can be p-methoxyphenyl—(PMP) or silylether—(e.g., tert-butyldimethylsilyloxy, OTBS).

The proton-coupled electron transfer, in some embodiments, is mediated by a photo-oxidant and Brønsted base. In some embodiments, the photo-oxidant is a transition metal catalyst. Suitable transition metal catalysts can be heteroleptic or homoleptic iridium complexes. For example, the photo-oxidant in a non-limiting embodiment can be [Ir(dF(CF₃)ppy)₂(5,5′-d(CF)bpy)]PF₆. In some embodiments, the photo-oxidant can also be an organic catalyst. For example, the photo-oxidant in a non-limiting embodiment can be 3,6-di-tert-butyl-9-(2,4,6-trimethylphenyl)-10-phenylacridinium tetrafluoroborate. The photo-oxidant can have any spectral absorbance properties consistent with the technical objectives herein. In some embodiments, the photo-oxidant absorbs radiation in the visible region of the electromagnetic spectrum. The photo-oxidant, for example, can absorb radiation having a wavelength of 400-600 nm, or 600-800 nm. In other embodiments, the photo-oxidant may absorb radiation in the ultra-violet or infrared regions of the electromagnetic spectrum.

The photo-oxidant can be present in the reaction mixture in any amount sufficient to achieve the desired depolymerization. In some embodiments, the amount of photo-oxidant is related to the number or concentration of hydroxyl groups on the polymer backbone or backbone segments. In some embodiments, the photo-oxidant is present in an amount of 0.01-10 mol. % or 0.5-5 mol % relative to mmol of the hydroxyl groups pendant to the backbone.

Further, any Brønsted base suitable for mediating proton coupled electron transfer can be used. The amount of Brønsted base can also be related to the number or concentration of hydroxyl groups on the polymer backbone or backbone segments. In some embodiments, the Brønsted base is present in the depolymerization reaction mixture in an amount of 0.1-3 equivalents relative to mmol of the hydroxyl groups pendant to the aliphatic backbone. Non-limiting examples of a base include collidine, collidine derivatives, and/or similar species. In another embodiment, the base can be tetrabutylphosphonium dimethylphosphate or similar species.

Alternatively, the proton-coupled electron transfer can be initiated via oxidation by an anode. For homogeneous depolymerization reactions, the hydroxylated polymer can interact directly with the anode using constant current or constant potential conditions. For heterogeneous depolymerization reactions, a redox mediator can be employed. The redox mediator is oxidized by the anode to form the oxidized state, which then mediates oxidation of the insoluble hydroxylated polymer substrate. A variety of organic mediators can be employed as the redox mediator, such as electron deficient triarylamines (E^{ox} approximately +1.67-1.90 V vs SCE in MeCN), which are sufficiently oxidizing to homolyze O—H bonds in the presence of a Brønsted base. Octahedral Ir(III) and Ru(II) complexes can also be used as electrochemical mediators because the ground state Ir(IV) and Ru(III) oxidation states of several Ir(III) and Ru(II) complexes are sufficiently oxidizing to mediate the desired O—H PCET process (E_{1/2}(Ir(IV)/Ir(III))=+1.69-1.94 V vs SCE in MeCN, E_{1/2}(Ru(III)/Ru(II))=+1.69-1.86 V vs SCE in MeCN).

Homolytic activation of O—H bonds of the hydroxyl groups induces formation of alkoxy radical intermediates followed by C—C bond β-scission events breaking the polymer backbone into depolymerization products. The depolymerization products can comprise alkyl radical intermediates reduced by hydrogen atom transfer. Accordingly, the reaction mixture can comprise a hydrogen atom donor (HAT) catalyst. The amount of HAT catalyst in the reaction mixture can be set relative to the amount or concentration of hydroxyl groups on the polymer backbone or backbone segments. In some embodiments, the HAT catalyst is present in an amount of 10-40 mol % relative to mmol of hydroxyl groups.

In some embodiments, the depolymerization products are converted into difunctionalized products containing functionality not originally present in the polymer or, alternatively, maintaining those derived from the polymer and/or derived from the depolymerization process. The difunctionalized products can subsequently be employed in repolymerization processes for the production of additional polymers. The difunctionalized products include α,ω-difunctionalized products.

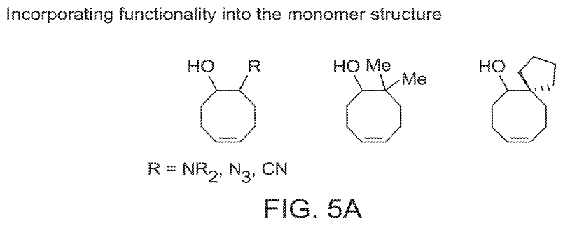

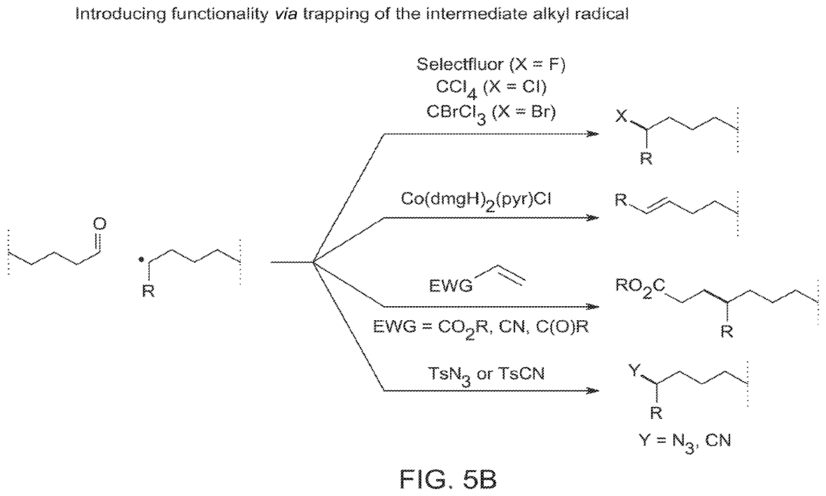

Given the large diversity of functionality tolerated by Ru-based olefin metathesis catalysts, access to α,ω-difunctional monomers bearing a broad array of functionalities can be achieved using depolymerization methods herein, potentially affording myriad high-value products. Two different strategies for incorporating functional groups into the products obtained through depolymerization can be employed: i) polymerizing monomers bearing the desired functionality or ii) installing the functionality onto the alkyl radical generated upon β-scission event. In terms of the first strategy, successful polymerization of cyclooctene monomers bearing amine, halogen, ester, and nitrile groups, among many others, is utilized. Incorporating these functional groups into the hydroxylated ROMP copolymers will permit their inclusion at the termini of the α,ω-difunctional products obtained post-degradation (FIG. 5A). Amine-substituted polymers can be used to produce degradation products that could be repolymerized to nylons. Nitrile groups could serve as a useful functional group on their own, being a key component in commercial materials, such as nitrile rubber, or be modified to a diverse array of functionalities through hydrolysis, reduction, and cycloadditions. Azides could also offer a means to access nitrogen containing degradation products. Tertiary alkyl groups could be incorporated into the monomer structure and provide an entry into the preparation of ω-branched products that otherwise would be challenging to synthesize.

The second approach involves functionalizing the alkyl radical generated upon β-scission (FIG. 5B). Halogenation can be performed through the incorporation of a halogenating agent, such as Selectfluor (for fluorination), carbon tetrachloride (for chlorination), and bromotrichloromethane (for bromination). Olefins can also be introduced at the termini of the post-degradation monomers through the action of a cobaloxime catalyst, which are effective hydrogen atom abstractors for the weak C—H bonds adjacent to radicals. Carbofunctionalization of the post-β-scission radical through the incorporation of Michael acceptors, such as acrylates and vinyl nitriles, can also be achieved. Additionally, the formation of C—N bonds from the post-β-scission radical through the addition of reagents such as TsN₃ or diisopropyl azodicarboxylate (DIAD), can afford useful precursors to nylon monomers. Moreover, TsCN can be used to directly introduce a nitrile group on the radical.

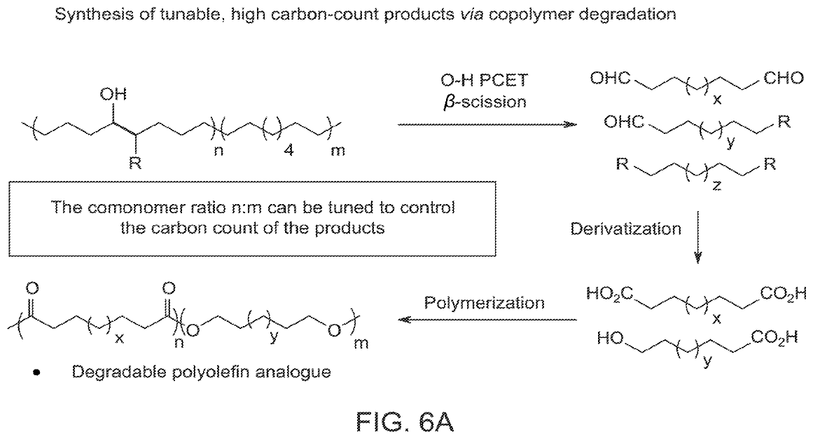

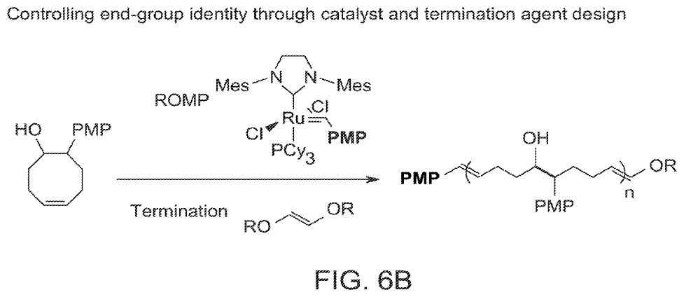

Moreover, the lack of convenient synthetic routes to the necessary long-chain α,ω-difunctional monomers has limited the potential of these degradable-by-design polyolefin analogues. Depolymerization of the copolymers of functionalized cyclooctenol and cyclooctene (COE) according to methods described herein can serve as a platform for the direct preparation of high-value α,ω-difunctional monomers with modular control over the length of the carbon chain. Copolymers of cyclooctenol and COE can be prepared over a range of comonomer ratios, thereby enabling control of the carbon count of the post-depolymerization products via modification of the ratio of cyclooctenol and COE monomers. Higher cyclooctenol:COE ratios allow the synthesis of shorter-chain α,ω-difunctional monomers, whereas lower ratios would lead to longer product monomers (FIG. 6A). The ability of this copolymerization/degradation platform to easily achieve the synthesis of the α,ω-difunctional oligomers with tunable chain lengths and end groups constitutes a new approach for selective oligomerization of ethylene, which is otherwise very challenging to achieve. Other copolymers can also be prepared with the goal of targeting a specific carbon count in the degradation products as required for a particular application (e.g., diesel fuel, waxes, polycondensation monomers). A key consideration in ensuring control over the carbon count of the post-degradation products is the distribution of the comonomers. Ideally, the hydroxylated comonomer will be distributed as uniformly as possible along the copolymer backbone so that the degradation reaction will provide access to a well-defined and narrow set of products.

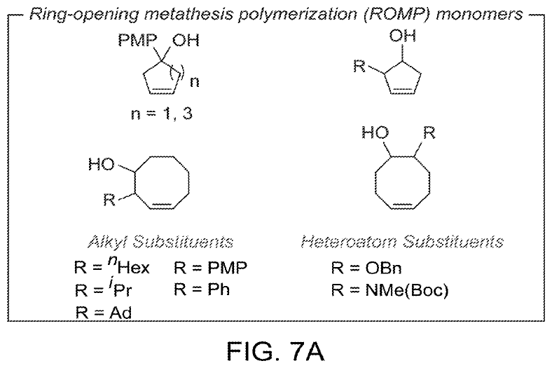

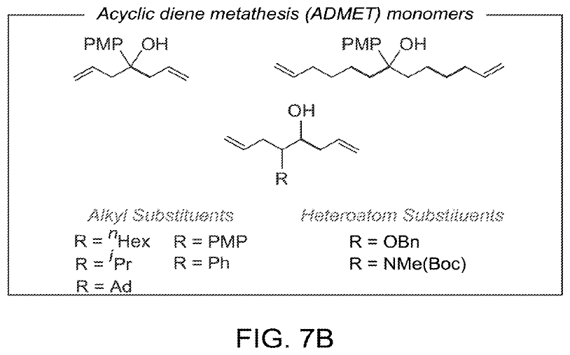

Another important consideration in the design of these upcyclable copolymers is the identity of the end group, which becomes increasingly relevant in the product distribution at low loadings of cyclooctenol comonomer, where each copolymer may degrade fully to only ten or so different products. To impart control over the end group identity, alternative catalyst structures are employed through modification of the Ru alkylidene, such as via the incorporation of a p-methoxyphenyl-containing benzylidene (FIG. 6B). Polymerization from such a catalyst can install a p-methoxyphenyl (PMP) group as the end group to ensure greater consistency with the other products obtained from depolymerizing copolymers comprising COE. Other alkylidene substituents can also be incorporated into the catalyst structure for polymers bearing other radical-stabilizing groups. Control over the chain end introduced upon termination can arise from the use of a terminating agent derived from alkyl vinyl ether, such as ethyl vinyl ether or butyl vinyl ether, which would install a hydroxyl group as a chain end. The design of this terminating agent is based on the use of ethyl vinyl ether to remove metathesis catalysts from polymer chain ends as inactivated Fischer carbene complexes. The resulting enol ether end group can then be hydrolyzed to provide an aldehyde product. Additionally, a suite of homopolymer materials can be constructed by incorporating other substituents within the cyclooctenol monomer (FIG. 7A).

Besides changes to monomer design, polymerization methods other than ROMP could be fruitful in the synthesis of polymers degradable via O—H PCET methods described herein. For example, acyclic diene metathesis (ADMET) allows the polymerization of linear α,ω-dienes to polyolefins that can be functionalized to afford polyethylene analogues much like ROMP. However, the monomer scope for ADMET is in principle broader than that for ROMP because ring strain is not needed to thermodynamically drive the polymerization reaction. Therefore, it is possible to access polymers with spacing between hydroxyl groups that is not dictated by the ring size of sufficiently strained monomers (e.g., hydroxyl groups spaced every 6 or >8 carbons), which through depolymerization could give rise to value-added feedstocks for the preparation of Nylon-6,6 and other condensation polymers (FIG. 7B). Because the functional-group tolerant ruthenium metathesis catalysts employed in ROMP can also mediate ADMET, a broad array of functional groups can be incorporated into ADMET polymers, including halogens, esters, alcohols, and protected amines. The polymerization of symmetric α,ω-diene monomers (e.g., a monomer bearing a geminal PMP and hydroxyl) affords access to regioregular homo- and copolymers that provides a single product upon degradation.

Methods described herein can demonstrate efficient depolymerization of synthetic hydroxylated polymers and copolymers. In some embodiments, at least 80 percent of the hydroxyl groups of the backbone or backbone segments can be converted to aldehyde products. For example, at least 90-99 percent of the hydroxyl groups can be converted to aldehyde products with polymers bearing p-methoxyphenyl groups.

These and other embodiments are further illustrated in the following non-limiting examples.

### Example 1—Depolymerization of Synthetic Hydroxylated Polymers

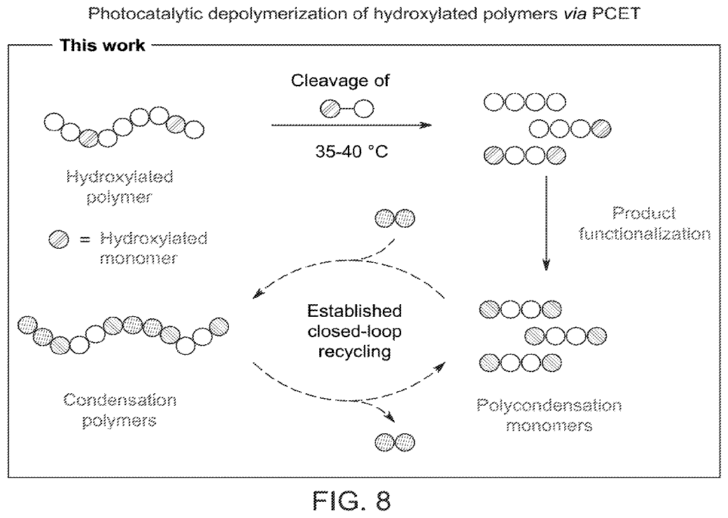

The present example demonstrated that PCET technology can be used to degrade a commercial poly(hydroxyl ether of bisphenol A) (i.e., phenoxy resin), epoxy thermosets, and novel polyolefin-type materials that are rendered degradable due to the inclusion of hydroxyl groups along the polymer backbone. (FIG. 1A). Notably, these depolymerization processes are compatible with insoluble polymeric materials, operate near ambient temperature, consume only visible photons, and afford well-defined and isolable products that can be further derivatized to produce monomers amenable to subsequent condensation polymerization (FIG. 8).

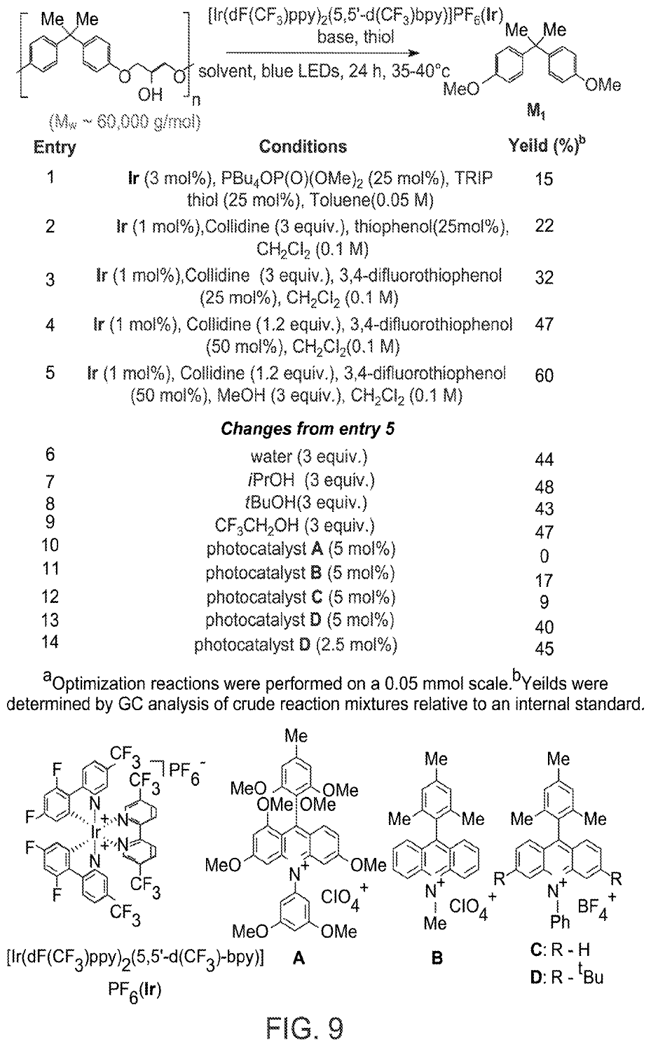

Initial evaluation focused on the depolymerization of the commercial poly(hydroxy ether of bisphenol A) (i.e., phenoxy resin) (FIG. 9). It was posited that the chain scission of this poly(hydroxy ether) would be favorable due to the stabilization of the C-centered radical by the adjacent oxygen atom within the backbone. The degradation of the phenoxy resin (M_{w}˜60,000 g/mol) was observed upon subjecting it to PCET methods described herein involving 3 mol % [Ir(dF(CF₃)ppy)₂(5,5′-d(CF₃)bpy)]PF₆ (Ir), 25 mol % tetrabutylphosphonium dimethylphosphate (dMe-phosphate), and 25 mol % 2,4,6-triisopropylbenzene thiol (TRIP thiol) in toluene (i.e., mol % relative to hydroxyl repeat unit). NMR analysis of the post-irradiation mixture revealed trace amounts of the expected aldehyde products and 18% yield of methoxybenzene-containing products. We also observed the formation of dimethyl bisphenol A (M₁) in 15% yield (based on gas chromatography (GC) quantification), which was confirmed by comparison with an authentic standard (FIG. 9, entry 1). These unexpected results were attributed to the instability of the desired 2-aryloxyacetaldehyde products under the polymer degradation conditions. To test this hypothesis, the fragmentation reaction of a model phenoxy dimer was studied under these conditions, which affords an aldehyde and a methoxybenzene-containing product. If both products are stable, a quantitative yield for each product would be observed. However, upon irradiation, this dimer was fragmented to afford the methoxybenzene-containing product in 98% yield while the aldehyde product was observed in only 21% yield, confirming that the latter is not stable under these conditions. The decomposition of 2-aryloxyacetaldehyde is postulated to proceed through the homolysis of the aldehyde C—H bond by a formal HAT event, affording a formyl radical that subsequently undergoes decarbonylation and H-atom abstraction with thiol to furnish monomer M₁.

Further optimization of the reaction conditions revealed that collidine, 3,4-difluorothiophenol, and dichloromethane were, respectively, the optimal base, HAT catalyst, and solvent for this degradation protocol (entries 2-4). Moreover, it was hypothesized that the use of an alcohol additive may facilitate the conversion of the initial 2-aryloxyacetaldehyde product to a hemiacetal intermediate, which could undergo a further C—C bond cleavage reaction through O—H PCET and β-scission to furnish M₁ as a final product. Accordingly, it was found that the inclusion of MeOH considerably improved the yield of M₁ to 60% (entry 5), whereas alternative conditions employing other alcohols or water were less effective (entries 6-9). Notably, the depolymerization was still effective when acridinium-based photocatalysts were used in place of Ir (entries 10-14). For example, the depolymerization of phenoxy resin in the presence of 2.5 mol % photocatalyst D afforded M₁ in 45% yield (entry 14).

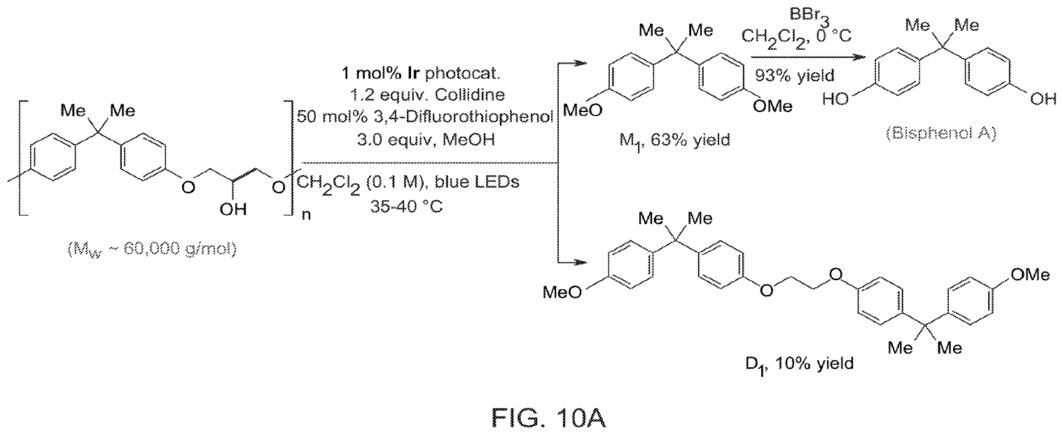

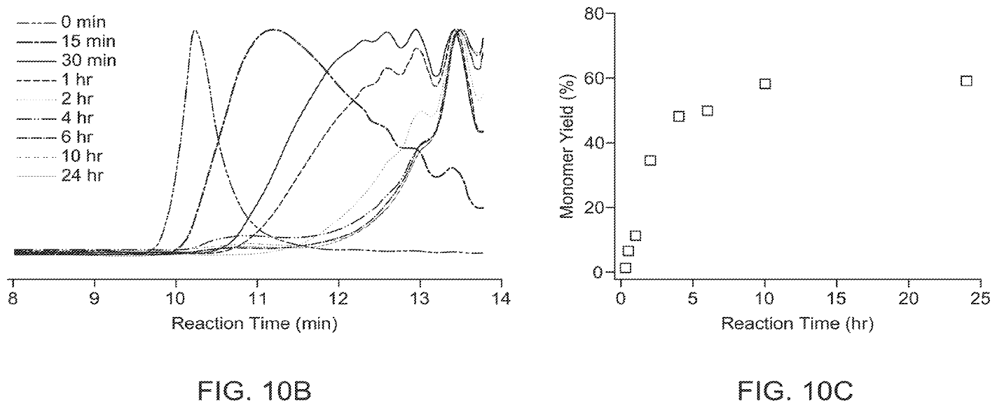

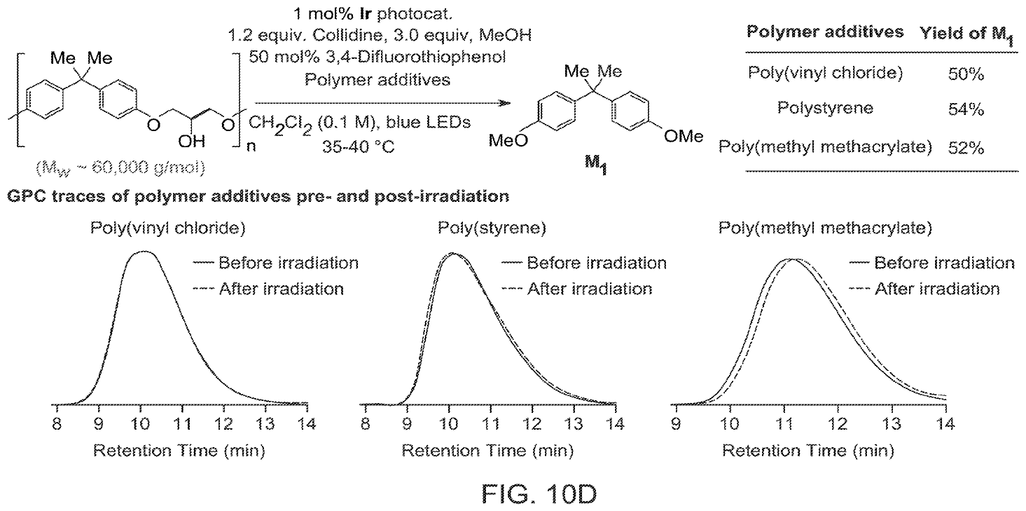

With the optimal conditions in hand, the monomeric product M₁ was isolated in an average yield of 63% on preparative scale (based on the theoretical yield of M₁ if the polymer is completely degraded), consistent with the observed GC yield (FIG. 10A). Formation of a dimer Di was also observed (10% isolated yield), presumably due to radical-radical homocoupling. We noted that the O-demethylation of M₁ with BBr₃ was also efficient, producing bisphenol A in 93% isolated yield. Altogether, efficient recycling of this commercial phenoxy resin was achieved, which is commonly used in coatings and adhesives, to bisphenol A, an important precursor to phenoxy/epoxy resin and many other commercial polymers, in an overall yield of 59% for the depolymerization/demethylation process. Overall, these results constitute the first example of a successful recycling platform for this phenoxy resin.

The extent of depolymerization of phenoxy resin was studied as a function of time (FIGS. 10B and 10C). Gel permeation chromatography (GPC) revealed a marked decrease in polymer molecular weight and concomitant appearance of smaller molecular weight peaks within the first hour of irradiation. Essentially complete polymer degradation was found to occur after only 4 hours, while the yield of M₁ continued to increase for 10 hours. These studies suggest that chain scission occurs rapidly under these light-driven PCET conditions and that the resulting oligomers are subsequently converted to a protected form of the monomer through iterative C—C bond cleavage steps.

Given that phenoxy resin is frequently used in the mixtures with other polymers, its depolymerization in the presence of different commodity plastics was investigated, including poly(vinylchloride) (M_{w}˜233,000 g/mol), polystyrene (M_{w}˜192,000 g/mol), and poly(methyl methacrylate) (M_{w}˜120,000 g/mol) (FIG. 10D). It was found that the presence of each of these materials did not significantly impact the degradation of phenoxy resin, as evidenced by the formation of M₁ in the average GC yields of 50-54% across the three additives GPC analysis of the non-hydroxylated polymers pre- and post-irradiation showed minimal change in their Mₙ or Ð, suggesting that they were not significantly degraded under these PCET conditions. These results highlight the chemoselectivity of the PCET activation for hydroxylated polymers and hold promise for the selective recycling of phenoxy resin in mixed waste streams.

Encouraged by these results, it was next sought to employ this PCET-driven degradation methodology to develop polyolefin-like materials that are degradable-by-design given that polyolefins are the largest class of polymers by global production. By incorporating a hydroxyl group within the cycloalkene monomer, we envisioned creating polyolefin mimics that could be degraded via our O—H PCET methodology. Consequently, hydroxylated polymers bearing a PMP- or OTBS-group were synthesized by polymerizing cyclooct-5-enols PMP and OTBS using a modified Grubbs second-generation (G2′) catalyst and subsequently hydrogenating the polyalkenes with diimide (FIG. 3B). These polymer design relied on the hypothesis that the favorability of C—C bond cleavage triggered by alkoxy radical is sensitive to the stability of the expelled radical. We anticipated that p-methoxyphenyl—(PMP-) or silylether—(OTBS-) substituent at the β-position could stabilize the C-centered radical formed upon β-scission and thereby enhance the efficiency of chain cleavage, permitting more efficient degradation.

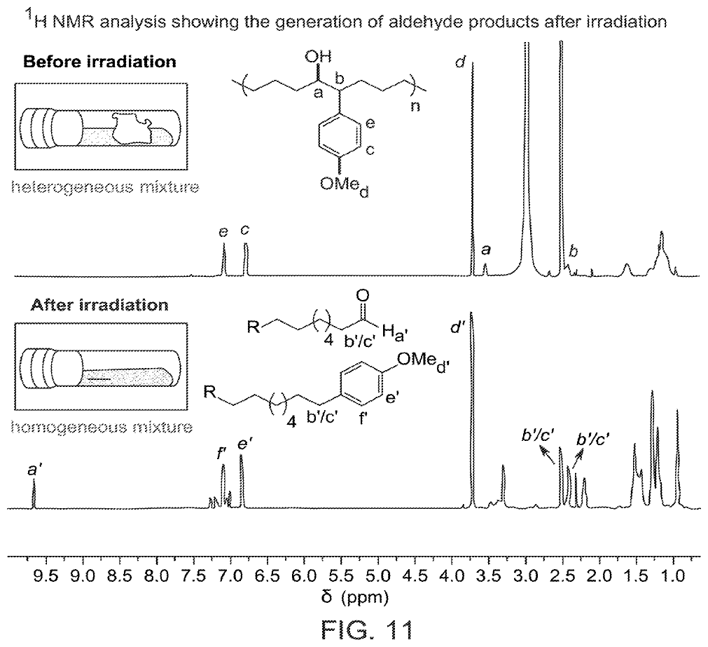

With these hydroxylated polymers derivatives in hand (referred to here as the PMP and OTBS homopolymer), it was aimed to see if our PCET methodology could be adapted to facilitate a controlled depolymerization. Blue light irradiation of the PMP homopolymer (Mₙ=8,400 g/mol, relative to polyethylene standard) in a toluene solution of 3 mol % Ir photocatalyst, 25 mol % dMe-phosphate, and 25 mol % TRIP thiol resulted in the formation of aldehyde products in nearly quantitative yield as assessed by ¹H NMR quantification, suggesting complete polymer degradation (FIG. 11). It should be noted that the reaction mixture prior to irradiation was heterogeneous as the PMP homopolymer is insoluble in toluene but became homogeneous as the reaction progressed. The success of depolymerization despite the heterogeneity of the reaction highlights the ability of this method to degrade insoluble polymeric materials. Additionally, GC and HPLC analysis showed the formation of the three expected monomeric products in excellent summative yields of 88%, including 1,8-octanedial (M₂), 8-(4-methoxyphenyl)octanal (M₃), and 1,8-bis(4-methoxyphenyl)octane (M₄), which were identified through comparison with authentic standards. The formation of these products suggests that the PMP homopolymer adopts a regioirregular structure, as suggested by previous studies on ROMP regioselectivity of similar 5-substituted cyclooctene monomers. Notably, the reaction efficiency did not seem to depend upon the Mₙ of the polymer, as there was no noticeable difference in the yield of monomeric products (88-91%, accessed via GC and HPLC quantifications) when PMP homopolymers with larger Mₙ (Mₙ=8,400-59,200 g/mol, relative to polyethylene standard) were used. It is also worth noting that the depolymerization was still effective if 1 mol % Ir photocatalyst or 5 mol % acridinium-based photocatalyst D was used, providing total yields of 80% and 70% of M₂-M₄ products, respectively.

Degradation of OTBS homopolymers was also efficient, although the total product yields were lower than those obtained from depolymerization reactions of PMP homopolymers. Upon blue light irradiation of these materials under the same catalytic conditions, the similarly insoluble OTBS homopolymers (Mₙ=2,400-112,100 g/mol, relative to polyethylene standard) were degraded to a mixture of soluble products in 42-72% yield via NMR quantification. GC analysis also revealed the formation of three expected monomers, including 1,8-octanedial (M₂), 8-((tert-butyldimethylsilyl)oxy)octanal (M₅), and 1,8-bis-((tert-butyldimethylsilyl)oxy)octane (M₆), in total yields of 9-25%. We noted a discrepancy in the yields quantified by NMR and GC, which we attributed to incomplete degradation of low molecular weight oligomers to monomeric products. However, this hypothesis could not be supported with GPC analysis, as the starting material proved insoluble in most solvents at room temperature. Notably, increasing the reaction time and catalyst loading did not further improve monomer yields.

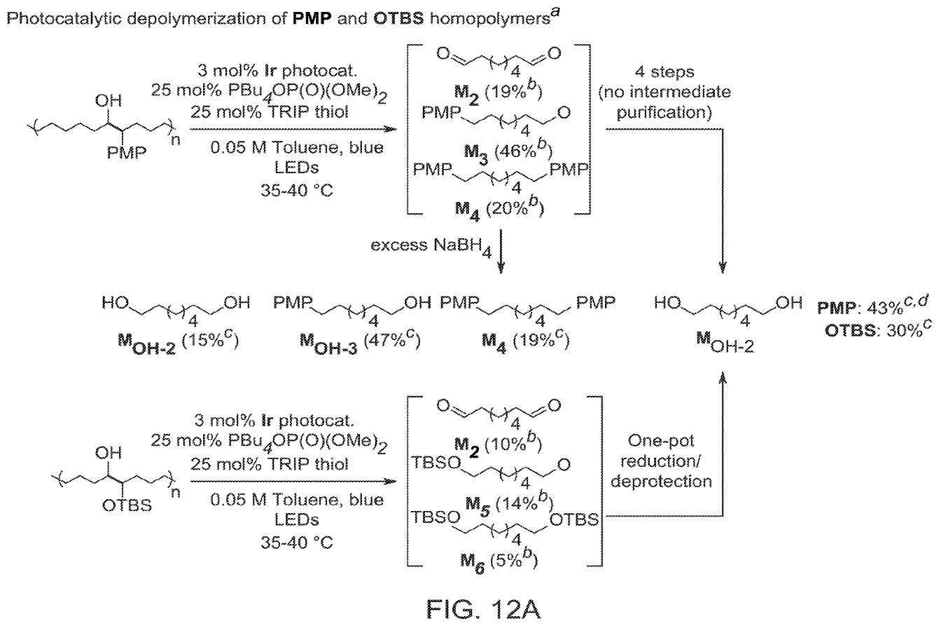

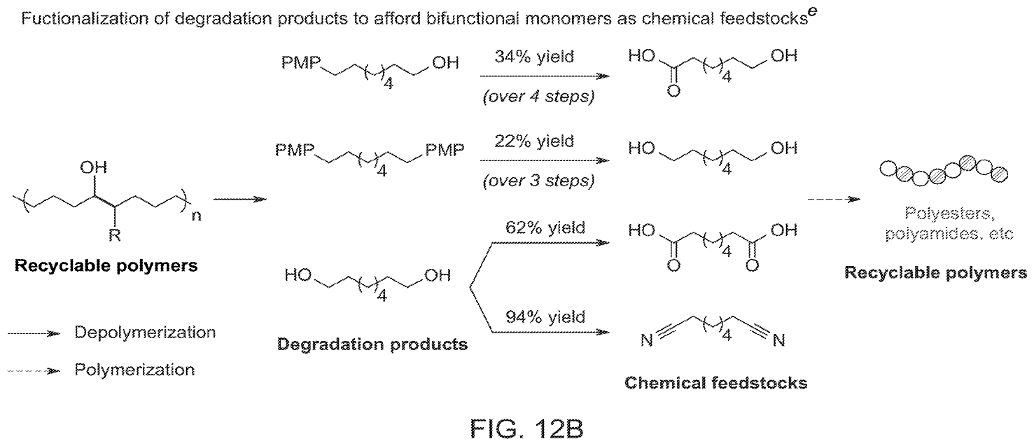

Having shown efficient degradation on analytical scale, it was sought to scale up the reaction and isolate the monomeric products from the crude depolymerization mixture. It was noticed that the aldehyde products were prone to partial autooxidation to the corresponding carboxylic acids during purification via silica gel chromatography. To circumvent this issue, a tandem reaction sequence was developed that converted the depolymerization product mixture into a single product and required only one purification step (FIG. 12A). Accordingly, treating the post-irradiation mixture of the PMP homopolymer to a 4-step reaction sequence-aldehyde reduction, benzylic oxidation, Baeyer-Villiger oxidation, and ester reduction-produced 1,8-octanediol as the sole product in an isolated yield of 43%. Similarly, the crude OTBS depolymerization mixture was subjected to a one-pot reduction/deprotection reaction, after which 1,8-octanediol was also isolated as the sole product in an average yield of 30%, which is in good agreement with the summative GC yields of M₂, M₅ and M₆.

In addition to developing processes to transform the mixtures of depolymerization products into a single isolated product, we also derivatized the depolymerization products into molecules that could serve as chemical feedstocks or monomers for subsequent condensation polymerization. To illustrate this process, the post-degradation mixture of PMP homopolymer was treated with excess sodium borohydride to convert the aldehyde products into the corresponding alcohols (M₂→M_{OH-2}, M₃→-M_{OH-3}) (FIG. 12A). These monomers (M_{OH-2}, M_{OH-3}, and M₄) were successfully isolated in 15%, 47%, and 19% yield, respectively (81% in total), in agreement with the observed GC/HPLC yields of M₂-M₄. These isolated products were then converted to a range of α,ω-difunctional monomers in moderate to excellent yields using established protocols. For example, 8-hydroxyoctanoic acid and 1,8-octanediol were obtained in overall yields of 34% and 22% from M_{OH-3} and M₄ through similar reaction sequences, both of which involved CAN-mediated benzylic oxidation and Baeyer-Villiger oxidation. In addition, 1,8-octanedinitrile-a precursor to 1,8-octanediamine-and 1,8-octanedioic acid (i.e., suberic acid) were also produced from M_{OH-2} in excellent yields (62% and 94%, respectively). Upon repolymerization, these α,ω-difunctional compounds would provide access to a wide range of condensation polymers (e.g., polyester, polyamide, and polycarbonate).

Having demonstrated the degradability of PMP and OTBS homopolymers, it was sought to investigate how the concentration of hydroxyl groups within the polymer backbone influences the material's degradability. A variety of copolymers were prepared (targeted total degree of polymerization=2000, referred to as PMP-x and OTBS-x, where x is the mol % of PMP (1a) or OTBS (1b), respectively) by polymerizing 1a or 1b with cyclooctene under the same conditions used for the synthesis of homopolymers. ¹H NMR analysis of the copolymerization reaction indicated the formation of statistical copolymers, as cyclooctene and the functionalized cyclooctenols (1a/1b) were consumed at similar rates during polymerization. The degradation of these copolymers under photocatalytic conditions was then studied and assessed the reaction efficiency via ¹H NMR quantification of the long-chain aldehyde products at high temperature (100° C.) due to their poor solubility at ambient temperature. It was found that PMP-x copolymers with at least 10 mol % of 1a (PMP-10, PMP-20, PMP-33, and PMP-50) underwent efficient degradation (i.e., 79-100%) (Table 1). Even copolymers with as little 1 mol % 1a were also significantly depolymerized (i.e., 43%). OTBS-x copolymers could also be degraded. While OTBS-1 was cleaved in good yield of 78%, depolymerization of OTBS-5 afforded the expected products in only 17% yield. Nonetheless, copolymers with at least 10 mol % 1b all degraded appreciably (29-77% yield). In principle, the long-chain aliphatic products of copolymer degradation can be derivatized into a wide range of α,ω-bifunctional compounds in a similar manner as described for monomers M₂-M₆. These macromonomers can subsequently be used for condensation polymerization to produce degradable materials that might exhibit similar thermomechanical properties as polyethylene. It is worth noting that the aliphatic chain length of these macromonomers is dependent on the original ratio of monomers present in the copolymerization reaction; copolymers with lower cyclooctenol loadings will produce longer-chain aliphatic products upon depolymerization. The statistical nature of this copolymerization offers a straightforward strategy to control and tune the thermomechanical properties of the recycled condensation polymers.

TABLE 1
Photocatalytic depolymerization reactions of PMP-x and OTBS-x copolymers^{a}

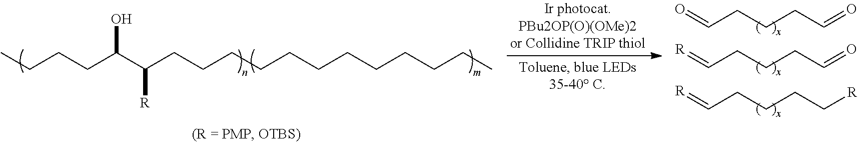

| Copolymer | 1 mol % | 5 mol % | 10 mol % | 20 mol % | 33 mol % | 50 mol % |
|---|---|---|---|---|---|---|
| PMP-x | 43% | 63% | 79% | >99% | >99% | 88% |
| OTBS-x | 78% | 17% | 34% | 29% | 73% | 77% |

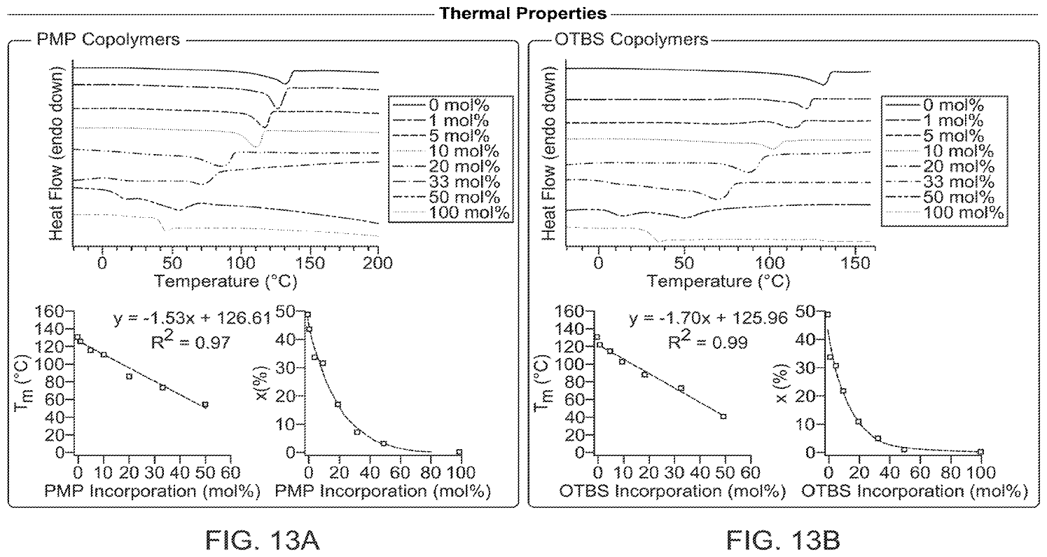

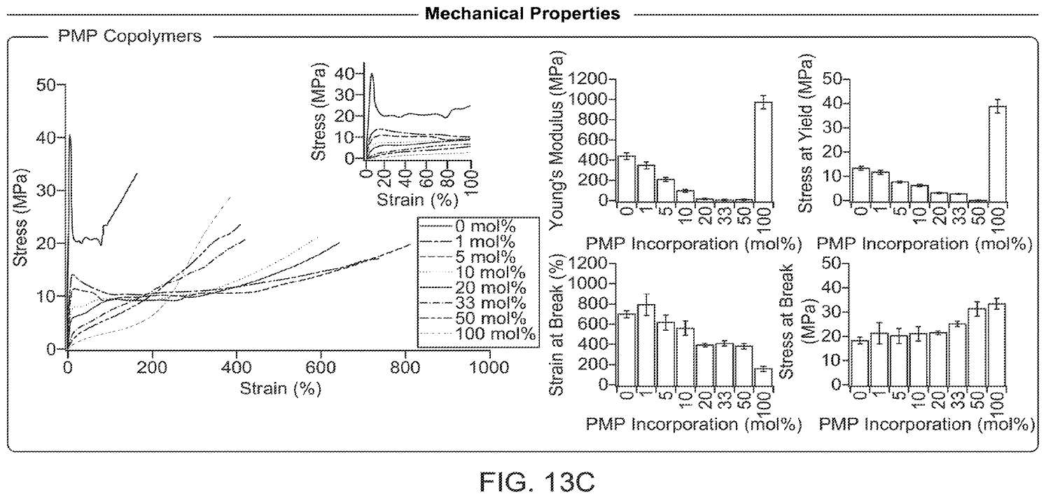

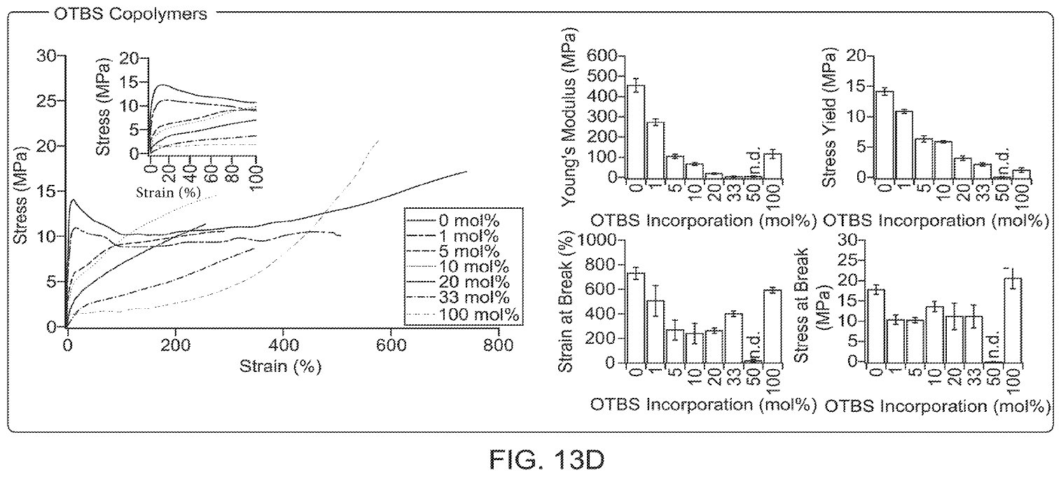

In addition to influencing the chain length of the degradation products, the loading of functionalized cyclooctenol monomers also modulates the physical properties of these materials. Specifically, the quantity and identity of these cyclooctenols (i.e., PMP or OTBS) have a significant impact on the materials' thermomechanical properties. The thermal properties of these materials was first studied through differential scanning calorimetry (DSC) and thermogravimetric analysis (TGA). Both PMP and OTBS homopolymers (FIGS. 13A and 13B, respectively) are amorphous materials with glass transition temperatures slightly above room temperature (T_{g}=43° C. and 31° C., respectively). In contrast, all PMP-x and OTBS-x copolymers exhibited a melt transition temperature (Tₘ), suggesting that they are semi-crystalline materials akin to unfunctionalized polyethylene (i.e., 0 mol % cyclooctenol). It was also observed that for both copolymer series, Tₘ decreases linearly and crystallinity (χ) decreases exponentially with increasing loading of PMP or OTBS. These trends are not impacted by the identity of the cyclooctenol monomer; both PMP and OTBS lower Tₘ and χ to the same extent at similar incorporation levels (mol %). This observation is in agreement with the Flory exclusion model, which posits that the quantity rather than the identity of substituents of sufficient size along polyethylene backbone impacts the crystallinity of the material. Notably, all the homopolymers and copolymers studied here are thermally stable as assessed by TGA, with most polymer samples exhibiting a decomposition temperature (T_{d,5%}) above 300° C.

The mechanical properties of these materials were also investigated through uniaxial tensile elongation testing. The PMP homopolymer (FIG. 13C) exhibited a distinct yield point (strain at yield (ε_{Y})=6.1±0.1%, stress at yield (σ_{Y})=40±3 MPa) that is accompanied by formation of a neck within the gauge length of the dog-bone specimen. A plateau in stress was observed following yielding until approximately 100% strain, during which the neck propagated throughout the entire gauge length. Further elongation of the specimen resulted in an increase in stress until break (strain at break (ε_{B})=170±20%, stress at break (σ_{B})=33±2 MPa). In contrast, the OTBS homopolymer (FIG. 13D) exhibited “rollover” yielding (ε_{Y}=8±2%, σ_{Y}=1.3±0.2 MPa) that was not accompanied by any neck formation and broke at a significantly higher strain and lower stress (ε_{B}=580∓20%, σ_{B}=20±3 MPa) than the PMP homopolymer. Despite being elongated to over 500% strain, the OTBS homopolymer relaxed to its original length after breaking. Moreover, compared to the PMP homopolymer, the OTBS homopolymer has a significantly lower Young's modulus (E=980±50 MPa versus E=120±20 MPa, respectively). Whereas the PMP homopolymer is a stiff and strong thermoplastic, the OTBS homopolymer is a flexible and ductile elastomeric material.

Collectively, these data suggest that the type of functional group within the cyclooctenol monomer regulates the mechanical properties of the corresponding materials. Incorporating these functionalized cyclooctenol units into the polyethylene backbone results in a decrease in Young's modulus (E) and yield strength (σ_{Y}) relative to unfunctionalized polyethylene (0 mol %). The quantity (mol %) rather than the identity (PMP vs. OTBS) of these functionalized cylooctenols seems to impact the stiffness (Young's modulus) and yielding behavior of these copolymer materials. In contrast, the identity of the functionalized cyclooctenols influenced the mechanical behavior of the PMP-x and OTBS-x copolymers following plastic deformation. In the OTBS-x copolymer series, stress at break (σ_{B}) and strain at break (ε_{B}) did not seem to depend on the mol % of OTBS. As the mol % of PMP increases in the PMP-x copolymer series, however, σ_{B} increases whereas ε_{B} decreases, suggesting an increase in strain hardening. These results show that the mechanical properties of these polyolefin derivatives can be systematically tuned by varying the quantity and identity of the functionalized cyclooctenol monomers PMP and OTBS used in copolymerization.

By copolymerizing different cyclooctenol monomers with COE, a library of polymeric materials was developed that exhibit a diverse yet predictable array of thermomechanical properties. Furthermore, because these hydroxylated polymers are degraded via the same mechanism, they can in principle be depolymerized together, thus facilitating the recycling of mixed plastic waste streams. To test the feasibility of a single PMP polymer waste stream, a mixture of PMP homopolymer and copolymers (i.e., equal masses of PMP, PMP-50, PMP-33, PMP-20, PMP-5, and PMP-1) was subjected to depolymerization conditions described herein. It was found that aldehyde products were generated in an average yield of 73% as assessed by high temperature ¹H NMR, suggesting that methods described herein are effective at depolymerizing a complex mixture of hydroxylated polymeric materials. Moreover, as in the case of phenoxy resin, this depolymerization methodology is not impacted by the presence of other polymeric additives. Conducting our standard depolymerization of PMP homopolymer in the presence of an equal mass of atactic polystyrene (M_{w}=192,000 g/mol) did not substantially impact the yield of depolymerization products; ¹H NMR analysis suggested a 92% yield of aldehyde products whereas GC and HPLC analysis showed the formation of the three expected products (M₂, M₃, and M₄) in summative yields of 78%. GPC analysis of the mixture pre- and post-irradiation showed no change in the Mₙ or Ð of the polystyrene additive.

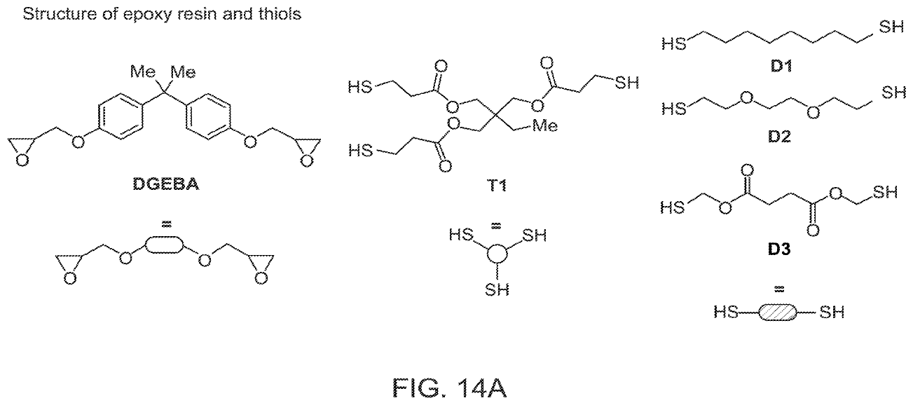

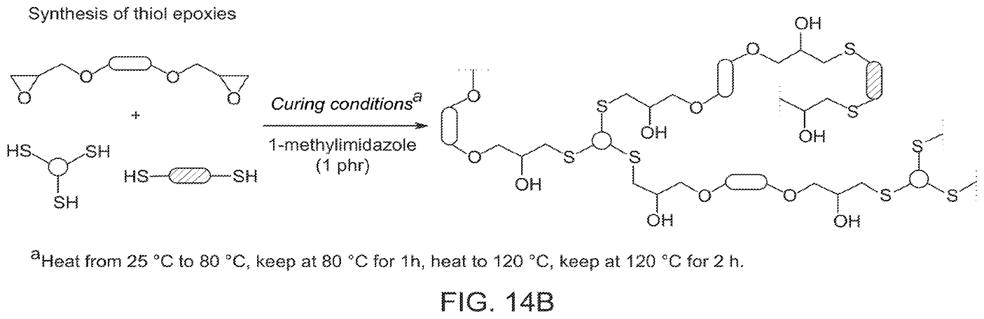

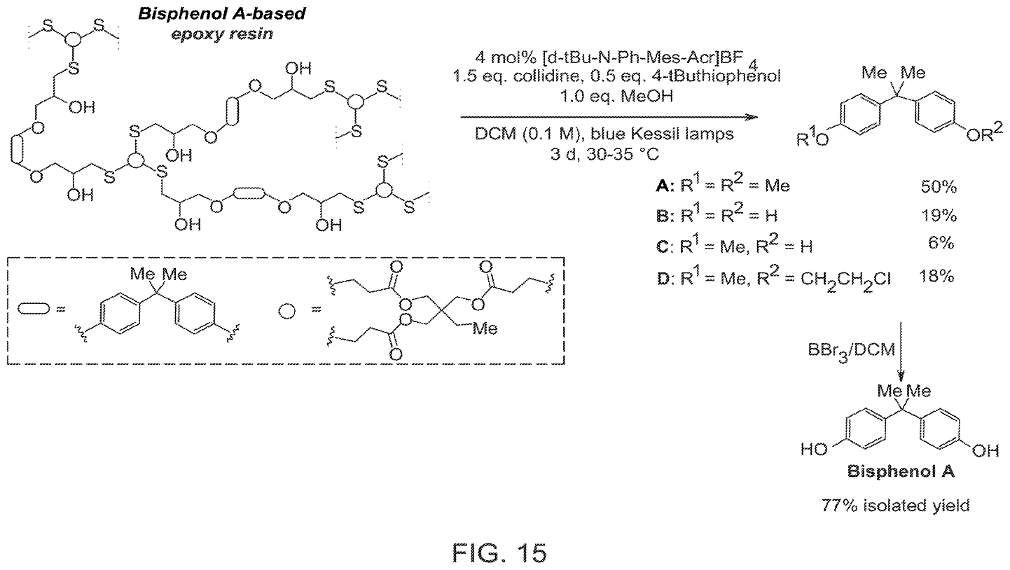

Lastly, it was also demonstrated that PCET technology can be used to degrade epoxy thermosets. As proof of concept, a wide range of thiol epoxies were synthesized from DGEBA (Aldrich, EEW=172-176 g/eq) and trimethylpropane tris(3-mercaptopropionate) (T1) (EW˜133 g/eq) in the presence of 1-methylimidazole catalyst (1 phr of the total mixture) (FIGS. 14A and 14B). To vary the crosslink density of these epoxies, different difunctional thiols (D1-3) with a wide range of loadings were also added to the curing mixtures. More specifically, the mole fractions of S—H groups in the dithiol additive with respect to the total S—H groups in both trithiol and dithiol are arbitrarily set to be 0%, 10%, 25%, and 50% in these formulations. Notably, the molar ratio of total thiols to epoxides is set to be 1:1, regardless of crosslink densities. Cured thermosets were then prepared by thoroughly mixing the samples, transferring to silicon molds, and keeping them in an oven for 1 h at 80° C. and 2 h at 120° C. (heating rate is set at 10° C./h) to ensure complete curing.

Next, the degradability of these epoxies under PCET conditions was investigated. After extensive investigation, it was found that these epoxies were completely degraded upon irradiating a solution of dichloromethane containing the finely ground epoxy, 4 mol % 9-mesityl-3,6-di-tert-butyl-10-phenylacridinium tetrafluoroborate (Mes-(d^{t}Bu)Ph-Acr⁺BF₄⁻), 1.5 equiv. collidine, 50 mol % 4-tert-butylthiophenol, and 1.0 equiv. MeOH (i.e., mol % relative to hydroxyl repeat unit). For all materials, the depolymerization produced a well-defined mixture of monomeric products in at least 90% total yields. Remarkably, even the parent DGEBA/T1 epoxy with the highest crosslink density was also completely degraded to provide monomeric products in a total yield of 93% via GC quantification (FIG. 15). Subsequent treatment of this product mixture with BBr₃ afforded bisphenol A in the overall isolated yield of 77%. Altogether, efficient recycling of the commercial epoxies was achieved, which is commonly used in high-performance coatings, adhesives, composites, and optical materials, to bisphenol A, an important precursor to phenoxy/epoxy resin and many other commercial polymers.

In conclusion, it was demonstrated a new strategy for the chemical recycling of a commercial hydroxylated polymers and novel degradable-by-design hydroxylated polyolefin polymers that relies on a PCET-based C—C bond cleavage reaction. The use of excited state catalysts provides the driving force necessary to enable these challenging C—C bond-breaking reactions, which would otherwise be thermodynamically disfavored at ambient temperatures. These depolymerization processes afford well-defined and isolable small molecule products, which can be further derivatized to produce monomers amenable to subsequent condensation polymerization. Moreover, the degradable-by-design polymers that we have developed exhibit a diverse yet tunable array of thermomechanical properties. Notably, these materials can be depolymerized together in a single plastic waste stream and in the presence of other types of polymeric materials. Overall, it is envisioned that this PCET methodology can be leveraged to enable the chemical recycling of other commercial polymeric materials and to develop next-generation degradable yet functional plastics.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A method of depolymerization comprising:
providing a synthetic polymer comprising a hydroxylated aliphatic backbone or hydroxylated backbone segments;
homolytically activating O—H bonds of the hydroxyl groups inducing formation of alkoxy radical intermediates followed by C—C bond β-scission events breaking the synthetic polymer backbone into depolymerization products.

2. The method of claim 1, wherein the depolymerization products comprise alkyl radical intermediates reduced by hydrogen atom transfer.

3. The method of claim 1, wherein the depolymerization products comprise difunctionalized products.

4. The method of claim 3, wherein the difunctionalized products comprise α,ω difunctionalized products.

5. The method of claim 1, wherein the homolytic activation occurs via proton-coupled electron transfer.

6. The method of claim 5, wherein the proton-coupled electron transfer is mediated by action of a photo-oxidant and a Bronsted base.

7. The method of claim 6, wherein the photo-oxidant is a transition metal catalyst.

8. The method of claim 7, wherein the transition metal catalyst is a heteroleptic or homoleptic iridium complex.

9. The method of claim 6, wherein the photo-oxidant is present in an amount of 0.01-10 mol. % relative to mmol of the hydroxyl groups pendant to the aliphatic backbone.

10. The method of claim 1, wherein the homolytic activation occurs via oxidation by an electrode.

11. The method of claim 1, wherein the homolytic activation occurs via oxidation of a redox partner by an electrode followed by proton-coupled electron transfer mediated by the oxidized redox partner.

12. The method of claim 1, wherein aliphatic backbone or backbone segments comprise pendant radical stabilizing moieties.

13. The method of claim 12, wherein the radical stabilizing moieties are at a β-positon to the hydroxyl groups.

14. The method of claim 12, wherein the pendant radical stabilizing moieties are selected from the group consisting of aryl, heteroaryl, and heteroatom.

15. The method of claim 1, wherein the polymer is a hydroxylated polyolefin.

16. The method of claim 15, wherein the synthetic polymer is hydroxylated polyethylene, hydroxylated polypropylene, or mixtures thereof.

17. The method of claim 3, wherein the difunctionalized products comprise polymerizable functionalities.

18. The method of claim 17, wherein the polymerizable functionalities are operable to form condensation polymers.

19. The method of claim 17, wherein the polymerizable functionalities are selected from the group consisting of carboxyl, hydroxyl, and nitrile.

20. The method of claim 3 further comprising polymerizing the difunctionalized products.

21. The method of claim 20, wherein polymerizing comprises condensation polymerization.

22. The method of claim 1, wherein providing the synthetic polymer comprises hydroxylating the aliphatic backbone or aliphatic backbone segments of an unhydroxylated polymer.

23. The method of claim 1, wherein providing the synthetic polymer comprises polymerizing hydroxylated monomer.

24. The method of claim 23, wherein the hydroxylated monomer comprises cycloalkene.

25. The method of claim 24, wherein the polymerization is ring opening metathesis polymerization.

26. The method of claim 23, wherein the hydroxylated monomer comprises one or more points of unsaturation.

27. The method of claim 26, wherein the polymerization is acyclic diene metathesis.

28. The method of claim 1, wherein at least 80 percent of the hydroxyl groups undergo homolysis.

29. The method of claim 1, wherein at least 90-99 percent of the hydroxyl groups undergo homolysis.

30. The method of claim 1, wherein the polymer is crosslinked.

31. The method of claim 25, wherein the polymer is an epoxy resin.
